(19) United States
(12) Patent Application Publication
(10) Pub. No.: US 2023/0064241 A1
(43) Pub. Date: Mar. 2, 2023

(54) MODULAR INCUBATION CHAMBER AND METHOD OF VIRUS INACTIVATION

(71) Applicant: MERCK PATENT GMBH, Darmstadt (DE)

(72) Inventors: Thomas Coton, IIIkirch-Graffenstaden (FR); Joseph William Muldoon, Berlin, MA (US); James Ormond, Belmont, MA (US)

(21) Appl. No.: 17/759,959

(22) PCT Filed: Feb. 3, 2020
(86) PCT No.: PCT/EP2020/052481

§ 371 Date: Aug. 2, 2022

(30) Foreign Application Priority Data
Feb. 3, 2020 (EP) 20305094.3

(51) Int. Cl. C12M 1/42; C12M 1/00; C12M 3/00

(52) U.S. Cl. CPC C12M 35/08; C12M 23/44; C12M 29/16

(57) ABSTRACT

An incubation chamber that may be provided in modular form in order to provide flexibility in flow rate and/or residence time of a product stream is disclosed. Assemblies including such incubation chambers for purification of biomolecules are also disclosed, as are methods for biomolecule purification, and in particular, methods for virus inactivation in an incubation chamber or in a plurality of incubation chambers arranged in series.

### BACKGROUND

This disclosure relates to the incubation of fluids with a large range of residence times and of flow rate(s). More particularly, embodiments of the incubation chambers and methods for incubation of fluids assist in achieving inline viral inactivation and ensuring that flow paths are set to create a narrow Residence Time Distribution (RTD) of the fluid particles.

Large-scale production and the economics around purification of therapeutic proteins, especially monoclonal antibodies is an increasingly important problem for the biopharmaceutical industry. Therapeutic proteins are generally produced in either mammalian cells or bacterial cells which have been engineered to produce the protein of interest. However, once produced, the protein of interest needs to be separated from various impurities such as host cell proteins (HCPs), endotoxins, viruses, DNA etc.

In a typical purification process, the cell culture harvest is subjected to a clarification step for removal of cell debris. The clarified cell culture harvest containing the protein of interest is then subjected to one or more chromatography steps, which may include an affinity chromatography step or a cation exchange chromatography step. In order to ensure viral safety of the therapeutic candidate and to comply with regulatory mandates, viral clearance unit operations are implemented into the purification process. Such steps include Protein A and ion exchange chromatography, filtration and low pH/chemical inactivation.

Virus inactivation is typically performed after a chromatography step (e.g. after affinity chromatography or after cation exchange chromatography). In a typical large scale purification process, the chromatographic elution pool containing the protein of interest is collected in a large tank or reservoir and subjected to a virus inactivation step/process for an extended period of time with mixing, which may take about 30 minutes to about several hours, in order to achieve complete inactivation of any viruses that may be present in the elution pool.

In monoclonal antibody (mAb) processing, for example, a sequence of independent unit operations is performed in batch mode, where holding tanks are used to store the material between unit operations and facilitate any necessary solution adjustments between steps. Typically, the material is collected into one tank where the material is adjusted to achieve the target inactivation conditions. This may be through the addition of acid to achieve a low pH target level or it may be through the addition of detergent in a detergent-based inactivation process. Next, the material is transferred to a second tank where it is held at the inactivation conditions for a specified incubation time. The purpose of the transfer is to eliminate risk of droplets on the walls of the first tank which may not have reached the target inactivation conditions and could contain virus particles. By transferring the material to a different tank, this risk is reduced.

Several virus inactivation techniques are known in the art, including exposing the protein solution to certain temperatures, pH’s, or radiation, and exposure to certain chemical agents such as detergents and/or salts. One virus inactivation process involves a large holding tank where material is held at inactivation conditions, such as low pH and/or exposure to detergent, for a given period of time, e.g., 60 minutes. This static hold step is a bottleneck in moving towards continuous processing.

Virus kill kinetics indicate, however, that the inactivation time may vary, which suggests that the processing time could be significantly reduced (or increased), the static holding tank for virus inactivation could be eliminated, and the method could be more amenable to continuous processing.

Recently, there has been a desire to have a continuous process where the unit operations are linked together and manual solution adjustments are minimized. To facilitate this, efforts are being made to develop in-line processing methods to enable in-line virus inactivation as well as other in-line solution adjustments. In continuous or semi-continuous flow systems, it would be desirable to provide a method of maintaining narrow residence time distributions in continuous flow systems.

Past practices of virus inactivation included introducing the liquid to be inactivated to a low pH solution (for example, under a pH of 3.6), homogenized if necessary, and then allowed to rest for a required period of time. The virus inactivation occurs then by the contact of the entire volume of liquid containing the virus with the low pH fluid in bulk and over a fixed dead processing time.

Continuous or semi-continuous processing requires that virus inactivation be done non-stop and in the context of meeting minimum residence times in order to assure effective inactivation of viruses without long dwell times at low pH conditions that could possible damage the products contained in the liquid (such as proteins) . These new systems require careful consideration of the residence times, the flow characteristics of the fluid (laminar vs. non-laminar), and the system/hardware requirements for achieving the residence times and flow characteristics.

Prior systems include continuous virus inactivation using irradiated light in a helically wound tube with centrifugal force acting on the fluid (WO2002038191, EP1339643B1, EP1464342B1, EP1914202A1 and EP1916224A1) and coil flow inverters (CFI) a tube is helically wound a number of turns around a coil axis where the number of turns and the angle of bends helps determine and improve the residence characteristics of the flow (as fluid is running along the chamber tubing, all of its particles do not flow at the same velocity due to viscous effects, shown especially near the tubing walls where the resulting velocity profile along the tube cross section which leads to a distribution of the residency time for the flow particles) (WO2015/135844A1), which are all hereby incorporated by reference. These systems are rigid and not designed to allow for flexibility of varying virus inactivation needs in various biopharmaceutical production processes.

Therefore, homogeneous mixing under predictable conditions with the most narrow Residence Time Distribution (RTD) of the fluid with the ability to use different flow rates and residence times in a continuous virus inactivation in a low pH setting is a particular goal. A new modular incubation chamber, which can be configured for a large range of residence times and flow rates would represent advance(s) in the art.

In continuous or semi-continuous flow systems, it therefore would be desirable to provide a method of maintaining narrow residence time distributions. Provision of a continuous or semi-continuous flow system for biomolecule purification would be desirable, particularly for protein purification.

### SUMMARY

Problems of the prior art have been addressed by embodiments disclosed herein, which relate to an incubation chamber that may be provided in modular form in order to provide flexibility in flow rate and/or residence time of a product stream while narrowing the dispersion of the residence time distribution. Assemblies including such incubation chambers for purification of biomolecules are also disclosed, as are methods for biomolecule purification, and in particular, methods for virus inactivation in an incubation chamber or in a plurality of incubation chambers arranged in modular form in series.

In certain embodiments, an incubation chamber is disclosed, comprising: an incubation chamber housing, the housing comprising an inlet port for receiving a fluid, an outlet port for dispensing the fluid, and an internal cavity; a helical coil positioned in the internal cavity and in fluid communication with the inlet port and the outlet port; wherein the incubation chamber has a top exterior surface having a first contour, and a bottom exterior surface having a second contour, wherein the top exterior surface can releasably mate or interconnect with an incubation chamber having a bottom exterior surface having the second contour, and the bottom exterior surface can releasably mate or interconnect with an incubation chamber having a top surface having the first contour.

In certain embodiments, an incubation chamber houses a helical or coiled tube. The chamber may have an inlet port in fluid communication with one end of the coiled tube, and an outlet port in fluid communication with another end of the coiled tube. Each port may be closed by an aseptic connector to form a closed system.

In certain embodiments, multiple incubation chambers may be vertically stackable and releasably interlockable. In some embodiments, stackable incubation chambers are interconnectable; they are in or may be placed in fluid communication with one another, e.g., the outlet of a first incubation chamber is in or is configured to be placed in fluid communication with the inlet of a second incubation chamber. In some embodiments, the outlet of the second incubation chamber is in or is configured to be placed in fluid communication with the inlet of a third incubation chamber, and so on.

In certain embodiments, the helical coil may have regions with different orientations in a single incubation chamber. In some embodiments, the helical coil may be stacked vertically in a single incubation chamber (i.e., stackable in the z-direction where the base of the incubation chamber is in the x-y plane).

In certain embodiments, the coiled tube is organized inside the incubation chamber to minimize the footprint of the incubation chamber.

In certain embodiments, a method for inactivating one or more viruses that may be present in a fluid sample containing a target molecule (e.g., an antibody or an Fc region containing protein) is provided, comprising subjecting the fluid sample to a chromatography process (e.g., affinity chromatography such as Protein A chromatography) or an ion exchange chromatography process to obtain an eluate; introducing a virus inactivation agent to said eluate; continuously introducing the eluate into an incubation chamber containing a helical flow channel and causing the eluate to flow in the flow channel for a time sufficient to inactive virus. In certain embodiments, the chromatography process is carried out in a continuous or semi-continuous mode. The eluate from the affinity chromatography process can be a real time elution from a column entering the system with all of its gradients of pH, conductivity, concentration, etc., or can be a pool of elution then subjected to inactivation after homogenization.

In some embodiments, the different process steps are connected to be operated in a continuous or semi-continuous manner. In some embodiments, a virus inactivation method, as described herein, constitutes a process step in a continuous or semi-continuous purification process, where a sample flows continuously from, for example, a Protein A affinity chromatography step or an ion-exchange chromatography step to the virus inactivation step to the next step in the process, which is typically a flow-through purification process step.

In some embodiments, the virus inactivation process step is performed continuously or semi-continuously, i.e., the eluate from the previous process step, such as the previous bind and elute chromatography step (e.g., Protein A affinity chromatography) flows into the virus inactivation step, which employs a fluid flow path contained in an incubation chamber, after which in some embodiments the virus inactivated eluate may be collected in a storage vessel until the next process step is performed, or in some embodiments may be fed directly and continuously to the next downstream process step.

Methods and apparatus described herein are able to achieve virus inactivation in a continuous or semi-continuous manner, which can provide significantly flexibility in flow rates and residence times due to the modular format.

### BRIEF DESCRIPTION OF THE DRAWINGS

FIG. 1 is a perspective view of an incubation chamber in accordance with certain embodiments;

FIG. 1A is a perspective view of an incubation chamber base and cover, in an open position, with a helical coil positioned in the internal cavity of the chamber, in accordance with certain embodiments;

FIG. 1B is another perspective view of an incubation chamber base and cover, in an open position, with a helical coil positioned in the internal cavity of the chamber, in accordance with certain embodiments;

FIG. 1C is yet another perspective view of an incubation chamber base and cover, in an open position, with a helical coil positioned in the internal cavity of the chamber, in accordance with certain embodiments;

FIG. 1D is yet another perspective view of an incubation chamber base and cover, in an open position, with a helical coil positioned in the internal cavity, in accordance with certain embodiments;

FIG. 2 is an exploded view of the incubation chamber of FIG. 1, with the internal chamber containing a helical coil in accordance with certain embodiments;

FIG. 3 is a top view of the internal chamber of the incubation chamber of FIG. 2 in accordance with certain embodiments;

FIG. 4 is a top view of a helical coil configured for placement inside an incubation chamber in accordance with certain embodiments;

FIG. 5 is a perspective view of an incubation chamber housing two layers of helical coil in accordance with certain embodiments;

FIG. 6 is a perspective view of a skewed helical coil in accordance with certain embodiments;

FIG. 7 is a top view of an incubation chamber showing the internal cavity with a skewed helical coil making optimum use of the space in the internal cavity in accordance with certain embodiments;

FIGS. 8A and 8B are perspective views of modular assemblies assembled on a movable cart;

FIG. 9 is a further perspective view of a modular assembly on a movable cart in accordance with certain embodiments;

FIG. 10 is a schematic view of a stackable cylindrical modular assembly in accordance with certain embodiments; and

FIG. 11 is a bottom perspective view of an incubation chamber in accordance with certain embodiments.

### DETAILED DESCRIPTION

A more complete understanding of the components, processes and devices disclosed herein can be obtained by reference to the accompanying drawings. The figures are merely schematic representations based on convenience and the ease of demonstrating the present disclosure, and is, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

The term “virus inactivation” or “viral inactivation” refers to the treatment of a sample containing one or more viruses in a manner such that the one or more viruses are no longer able to replicate or are rendered inactive. In methods described herein, the terms “virus” and “viral” may be used interchangeably. Virus inactivation may be achieved by physical means, e.g., heat, ultraviolet light, ultrasonic vibration, or using chemical means, e.g. pH change or addition of a chemical (e.g., detergent). Virus inactivation is typically a process step which is used during most mammalian protein purification processes, especially in case of purification of therapeutic proteins from mammalian derived expression systems. In methods described herein, virus inactivation is performed in a fluid flow channel. It is understood that failure to detect one or more viruses in a sample using standard assays known in the art and those described herein, is indicative of complete inactivation of the one or more viruses following treatment of the sample with one or more virus inactivating agents. A virus inactivating agent, as used herein, may include a solution condition change (e.g. pH, conductivity, temperature, etc.) or the addition of a detergent, a salt, an acid (e.g., acetic acid, with a molarity to achieve a pH of 3.6 or 3.7), a polymer, a solvent, a small molecule, a drug molecule or any other suitable entity, etc., or any combination thereof, which interacts with one or more viruses in a sample, or a physical means (e.g., exposure to UV light, vibration etc.), such that exposure to the virus inactivating agent renders one or more viruses inactive or incapable of replicating. In a particular embodiment, a virus inactivation agent is a pH change, where the virus inactivating agent is mixed with a sample containing a target molecule (e.g., an eluate from a Protein A bind and elute chromatography step) in a static mixer, for example, and then directed to a flow channel.

The term “continuous process” as used herein, includes a process for purifying a target molecule, which includes two or more process steps (or unit operations), such that the output from one process step flows directly into the next process step in the process, without interruption, and where two or more process steps can be performed concurrently for at least a portion of their duration. In other words, in the case of a continuous process, it is not necessary to complete a process step before the next process step is started, as long as a portion of the sample is always moving through the process steps.

Similarly, a “semi-continuous process” may encompass an operation performed in a continuous mode for a set period of time with periodic interruption of one or more unit operations. For example, stopping the loading of feed to allow for the completion of other rate-limiting steps during a continuous capture operation.

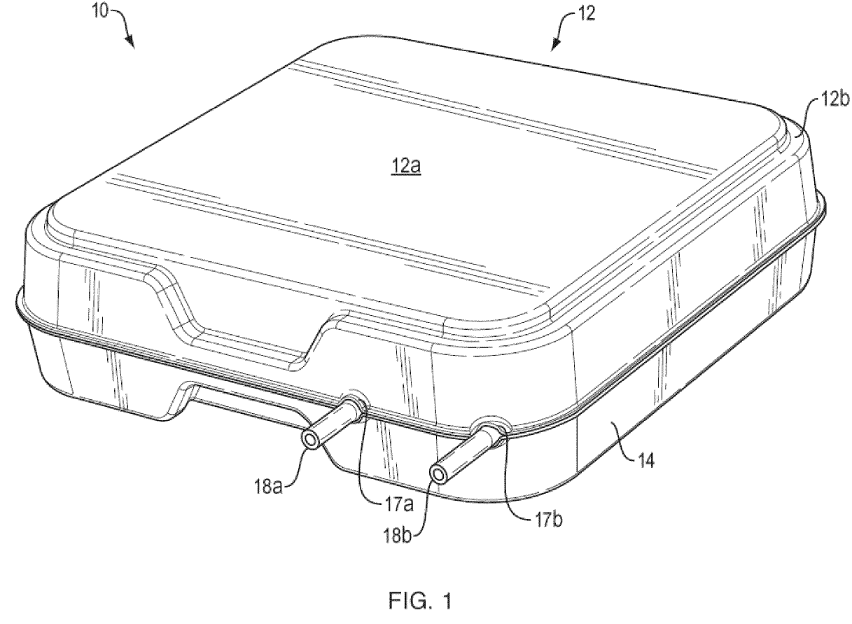

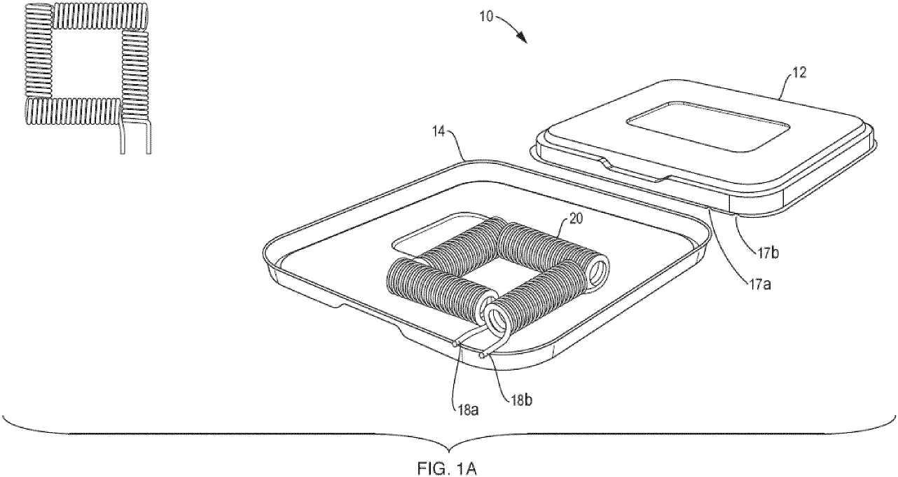

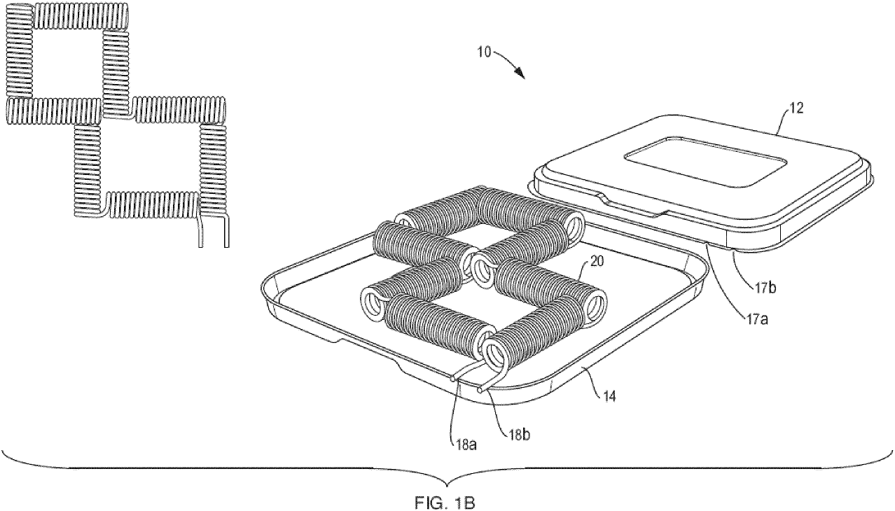

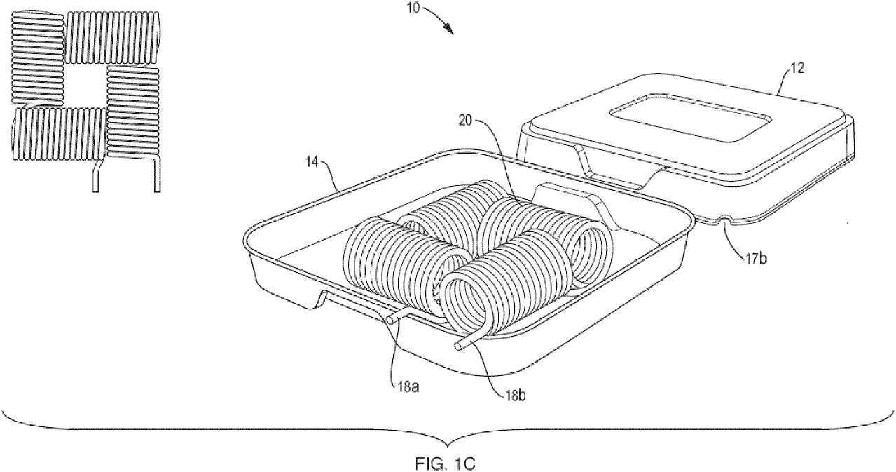

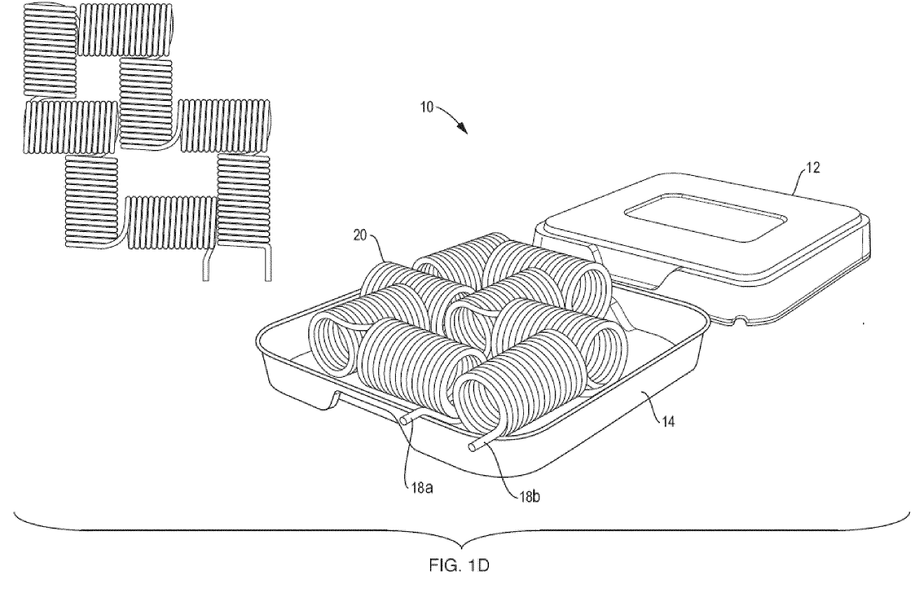

Turning now to FIGS. 1-4, there is seen an incubation chamber 10 in accordance with certain embodiments. In the embodiment shown, the incubation chamber 10 has a cover 12 and a base 14. The cover 12 and base 14 may be configured to mate in sealing relation to enclose an internal incubation chamber cavity 16 (FIG. 2). In some embodiments, the cover 12 may be hinged to the base 14. A pair of apertures 17a, 17b may be provided in the incubation chamber to accommodate an inlet end 18a and an outlet end 18b of helical coil 20. In the embodiment shown, the apertures (and inlet end 18a and outlet end 18b of helical coil 20) are positioned at the same side of the incubation chamber 10, but those skilled in the art will appreciate that the arrangement of the helical coil 20 in the incubation chamber 10 can be modified such that the inlet end 18a and outlet end 18b are located at different sides of the incubation chamber 10. One suitable inside diameter of the helical tube 20 in this embodiment is 9.6 mm, with a tube length of 28.08 m.

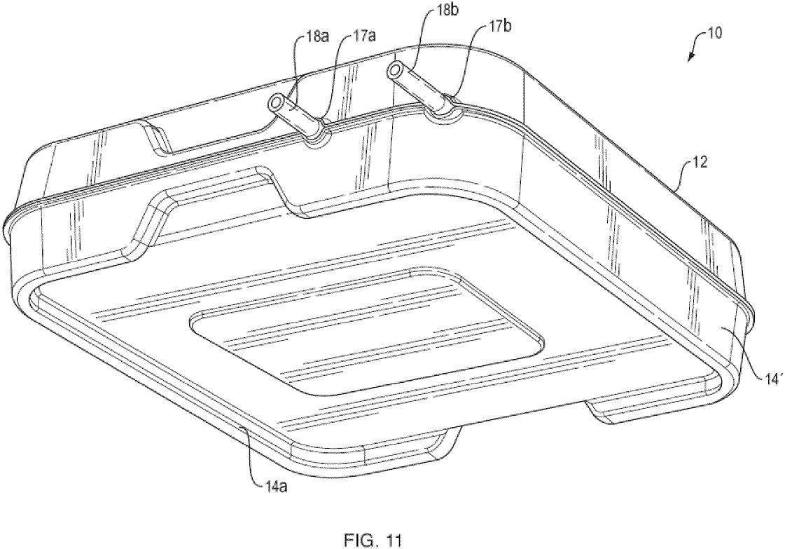

In certain embodiments, top or uppermost surface or face 12a of cover 12 may have a contour configured to mate with a corresponding contour of the bottom surface or face (not shown) of a base 14' of another incubation chamber 10', so that a plurality of incubation chambers 10 are easily stackable and interlockable. For example, in the embodiment shown, the cover 12 has a perimeter edge 12b that is lower in height than the top or uppermost surface 12a. The perimeter edge 12b can accommodate a corresponding perimeter flange 14a (FIG. 11) that extends downwardly or axially from a base 14' such that the flange sits in the recess allowing two incubation chambers 10 to mate or interlock in stacking relation. In some embodiments the respective configurations of the top and bottom surfaces are such that they may be easily removed from one another after interlocking.

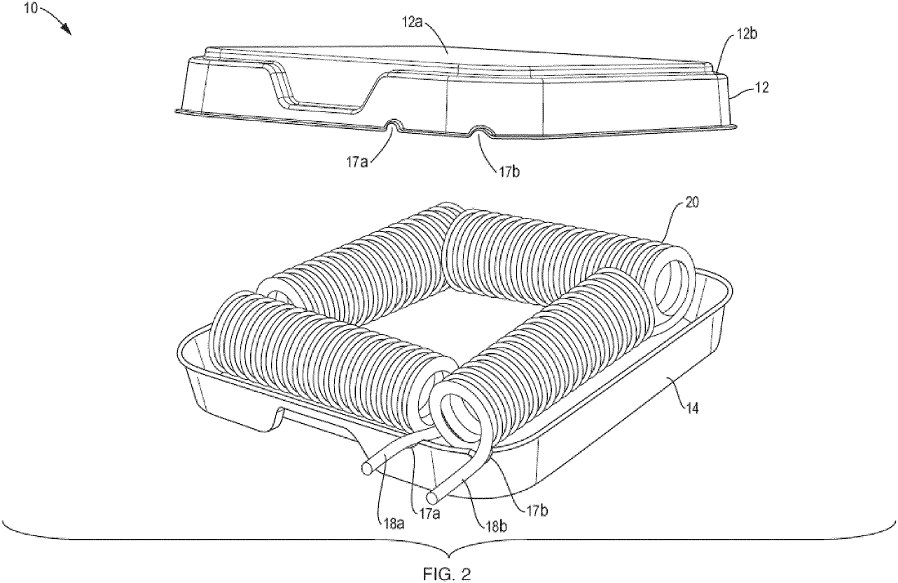

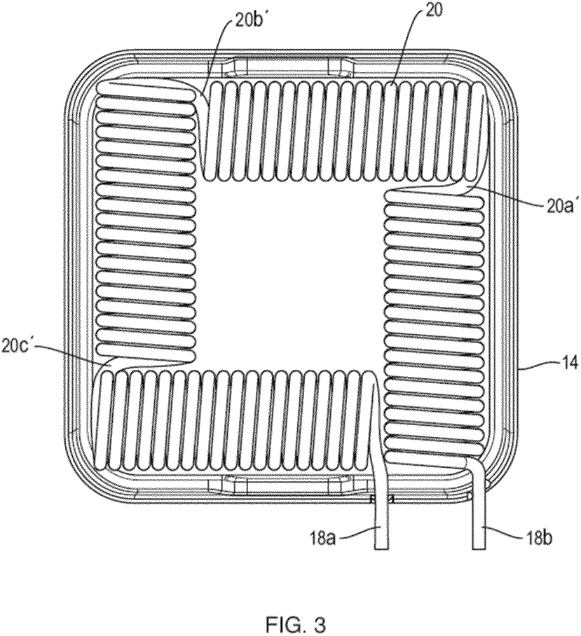

Turning now to FIG. 4, there is shown one embodiment of a helical coil 20 suitable for use in the incubation chamber 10. Preferably the helical coil 20 is made of gamma sterilizable material. For example, circular plastic tubing such as tubing made of silicone is suitable for the helical coil 20. In the embodiment shown, the helical coil 20 is arranged to enable an optimum fluid flow path in the incubation chamber 10. To that end, the helical coil has a first region 20a that is positioned in a “y” direction; a second region 20b that is positioned in an “x” direction that is orthogonal or substantially orthogonal to the first region 20a; a third region 20c that is positioned in an “x” direction that is orthogonal or substantially orthogonal to the second region 20b (and thus parallel or substantially parallel to the first region 20a); and a fourth region 20d that is orthogonal or substantially orthogonal to the third region 20c (and thus parallel or substantially parallel to the second region 20b). Certain regions include a coil transition portion that changes direction as it transitions from one coil region to the next. Thus first region 20a has a coil transition portion 20a' that transitions from first region 20a to second region 20b; second region 20b has a coil transition portion 20b' that transitions from second region 20b to third region 20c; and third region 20c has a coil transition portion 20c' that transitions from third region 20c to fourth region 20d. The helical coil 20 includes inlet end 18a and outlet end 18b and a continuous fluid flow path between them. Accordingly, each of the regions 20a, 20b, 20c and 20d is in fluid communication, defining the continuous fluid flow path. The so configured coil may be neatly positioned in an internal cavity of an incubation chamber 10 as shown in FIG. 3.

The number of winds in the helical coil 20 and the diameter of the helical coil 20 each can vary and are determined at least in part by the desired residence time of the sample in the flow path defined by the helical coil 20. Suitable design parameters include about 15-21 windings, a tube inside diameter of about 3.2-9.6 mm (⅛"-⅜"); and a winding core diameter of 40-105 mm. Preferably there are a minimum of 5 windings. In certain embodiments, the residence time should be sufficient time to enable virus inactivation at an effective pH to inactivate virus (e.g., maximum pH of 3.6 over at least about 30 minutes. Those skilled in the art appreciate that exposing the product sample to a sufficiently low pH to inactive viruses for too long a period can deteriorate the product, and thus the embodiments disclosed herein enable easy tailoring of the residence time and flow through the coil(s) in one or more incubation chamber(s) 10 to optimize virus inactivation. Typically the flow is laminar in the helical coil (e.g., Reynolds Number <2000), although a turbulent flow regime has no negative impact on the incubation chamber efficiency (the flow may even be turbulent in other parts of the system). The pH may be verified using sampling and offline sensors, for example.

FIG. 1A illustrates an embodiment of an incubation chamber 10 where the region defining the internal cavity is not completely occupied by a helical coil 20. In the embodiment shown, the helical coil 20 is positioned in a lower right quadrant of the internal cavity, closest to the apertures 17a and 17b, in order to minimize the straight lengths of the helical tube 20. In this embodiment, a suitable tube ID is 4.8 mm and a suitable tube length is 11.99 m.

FIG. 1B illustrates another embodiment of an incubation chamber 10 where the region defining the internal cavity is also not completely occupied by a helical coil 20. In the embodiment shown, the helical coil 20 is positioned in a lower right and upper left quadrant of the internal cavity, the lower right region being closest to the apertures 17a and 17b, in order to minimize the straight lengths of the helical tube 20. In this embodiment, a suitable tube ID is 4.8 mm and a suitable tube length is 23.99 m.

FIG. 1C illustrates yet another embodiment of an incubation chamber 10 where the region defining the internal cavity is not completely occupied by a helical coil 20. This embodiment is similar to that of FIG. 1A, except that the footprint of the incubation chamber 10 itself is larger (e.g., a height of 130 mm vs. a height of 65 mm for the incubation chamber of FIG. 1A), and thus the internal diameter of the helical tube 20 may be larger. In the embodiment shown, the helical coil 20 is positioned in a lower right quadrant of the internal cavity, closest to the apertures 17a and 17b, in order to minimize the straight lengths of the helical tube 20. In this embodiment, a suitable tube ID is 6.4 mm and a suitable tube length is 20.49 m.

FIG. 1D illustrates yet another embodiment of an incubation chamber 10 where the region defining the internal cavity is not completely occupied by a helical coil 20. This embodiment is similar to that of FIG. 1B, except that the footprint of the incubation chamber 10 itself is larger, and thus the internal diameter of the helical tube 20 may be larger. In the embodiment shown, the helical coil 20 is positioned in an upper left quadrant and lower right quadrant of the internal cavity, closest to the apertures 17a and 17b, in order to minimize the straight lengths of the helical tube 20. In this embodiment, a suitable tube ID is 6.4 mm and a suitable tube length is 40.98 m.

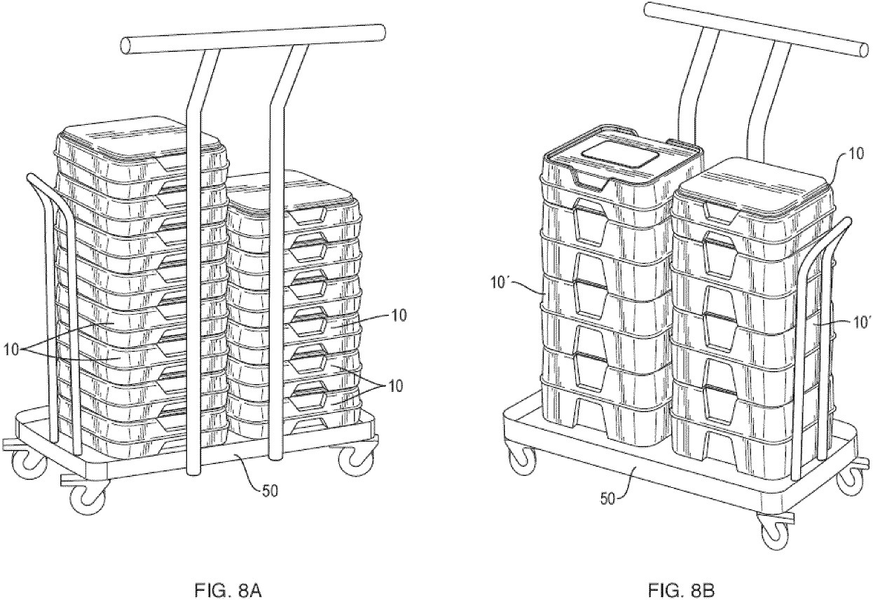

In certain embodiments, a plurality of incubation chambers may be arranged in series, such that the outlet of a first incubation chamber feeds into the inlet of a second incubation chamber, etc. A modular assembly thus can be built (e.g., FIGS. 8A and 8B), providing the user with flexibility to increase residences times and/or flow rates by adding one or more incubation chambers to the assembly, or decrease residence times and/or flow rates by removing one or more incubation chambers from the assembly. In certain embodiments, the connections between incubation chambers are aseptic connections (e.g., ASEPTIQUIK^{®} connectors) ensure a fully closed system. The assembly of modular incubation chambers may be supported on a cart 50 or the like (FIGS. 8A and 8B), which may be movable from place to place. The cart 50 may provide side access to the user to facilitate set up.

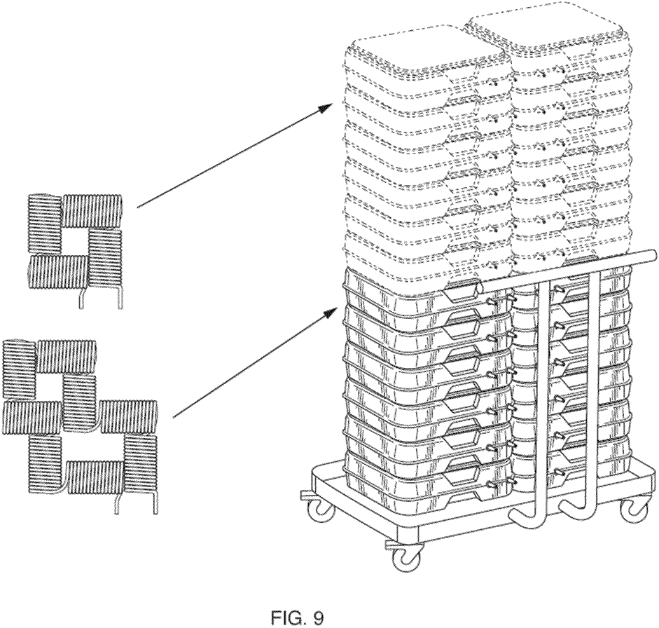

As best seen in FIG. 9, a modular assembly of fluidly connected incubation chambers can include incubation chambers of different sizes. For example, in the embodiment shown, there are 24 incubation chambers with the helical coil 20 configuration of FIG. 1C, and 12 incubation chambers with the helical coil 20 configuration of FIG. 1D.

Alternatively or in addition, higher flow rates and/or longer residence times can be achieved by using incubation chambers 10 having helical coils with a greater inner diameter. Similarly, lower flow rates and/or shorter residence times can be achieved by using incubation chambers having helical coils with a smaller inner diameter.

In some embodiments, a modular assembly may be formed where the helical coil length and/or diameter (inside diameter) in all of the incubation chambers of the modular assembly is the same. In some embodiments, a modular assembly may be formed where the helical coil length and/or diameter in one or more incubation chambers of the assembly is different from the helical length and/or coil diameter in one or more other incubation chambers of the assembly. In some embodiments, a modular assembly can be formed of a plurality of incubation chambers fluidly connected or connectable where at least one incubation chamber in the plurality has a helical tube length twice that of the helical tube length in another incubation chamber in the plurality.

Accordingly, residence time and flow rate flexibility is achieved without having to modify the external dimensions of an incubation chamber 10.

The flow rate and tubing length may be selected to target a particular residence time. For virus inactivation applications, the residence time may be chosen as the time sufficient to achieve virus inactivation within the flow channel, preferably with some safety factor. For example, in an embodiment where a residence time of 30 minutes is sufficient for virus inactivation, design parameters can be chosen so that the bulk of the particles will pass through the chamber in about 36 minutes, with the fastest residing in the chamber for about 32 minutes, and the slowest for about 45 minutes. The minimum residence time may also depend on regulatory guidance in terms of an acceptable safety factor for virus inactivation.

Suitable nominal residence times include, but are not limited to, 10 minutes, 15 minutes, 20 minutes, 25 minutes, 30 minutes, 35 minutes, 40 minutes, 45 minutes, 50 minutes, 55 minutes and 60 minutes.

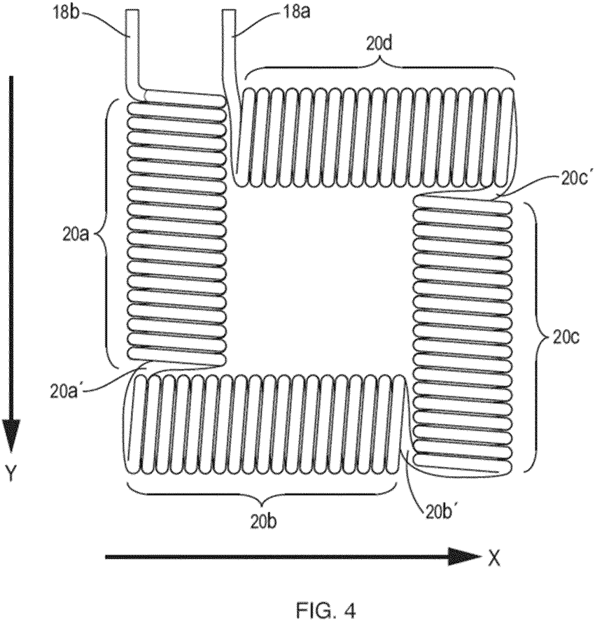

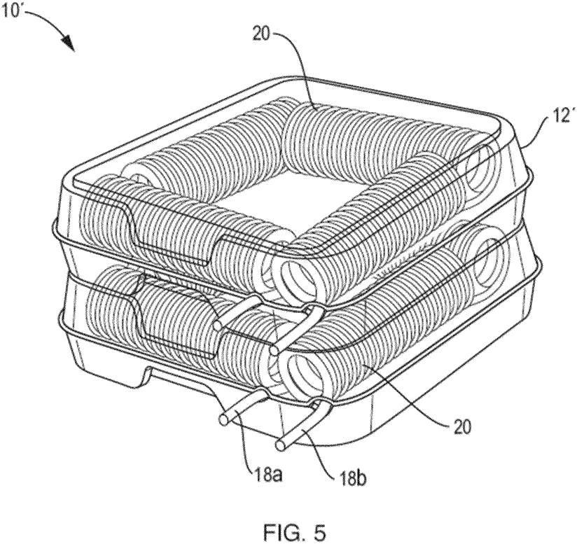

As shown in FIG. 5, in some embodiments the internal cavity of an incubation chamber 10' is deep enough, e.g., has a sufficient volume to house two or more vertical layers of helical coils 20. In some embodiments, such incubation chambers 10' can be used in conjunction with incubation chambers 10, as shown in FIG. 8B.

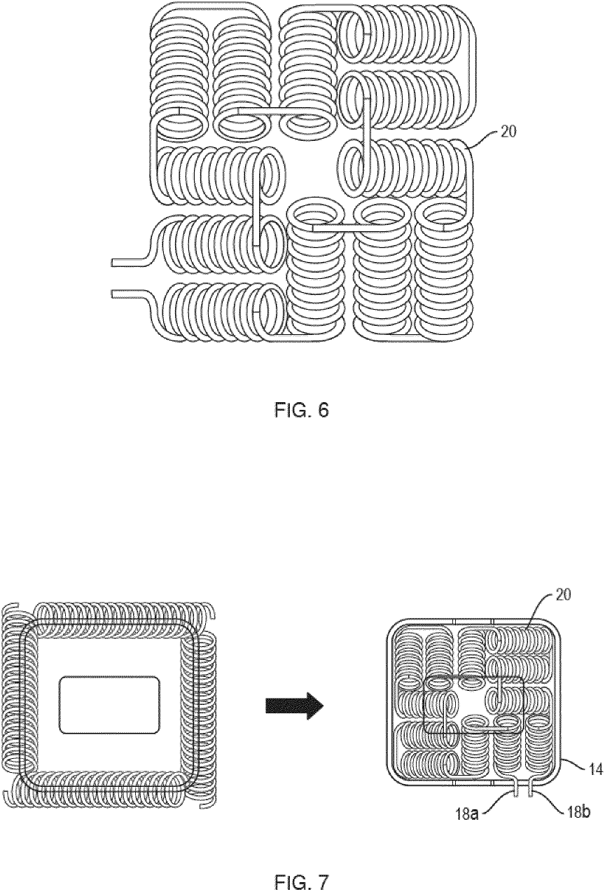

In some embodiments, in order to decrease the height of an incubation chamber 10, the helical coil 20 may be formed in a skewed configuration as shown in FIG. 6. Suitable skew angles range from about 30° to about 60° While this adds to the overall width of an incubation chamber, the footprint of the incubation chamber 10' or chambers can be minimized by having the helical coil occupy as much internal space within the incubation chamber as possible. One suitable arrangement of skewed helical coil is illustrated in FIG. 7.

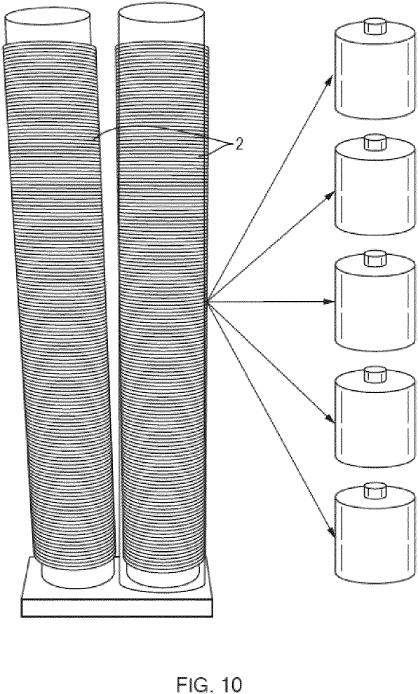

In a still further embodiment, the incubation chamber 10 may be in the form of a cylinder, with the helical coil flow path coiled around a cylindrical core. Such cylinders may be stackable as shown in FIG. 10, and arranged in series in a manner similar to the cassette-like embodiment of FIG. 1.

What is claimed is:

1. An incubation chamber, comprising:
an incubation chamber housing, comprising:
an inlet port for receiving a fluid,
an outlet port for dispensing the fluid, and
an internal cavity;
a helical coil positioned in said internal cavity and in fluid communication with the inlet port and the outlet port;
wherein said incubation chamber has a top exterior surface having a first contour, and a bottom exterior surface having a second contour, wherein said top exterior surface can releasably mate with an incubation chamber having a bottom exterior surface having said second contour, and said bottom exterior surface can releasably mate with an incubation chamber having a top surface having said first contour.

2. The incubation chamber of claim 1, wherein said helical coil is skewed.

3. The incubation chamber of claim 1, wherein said helical coil has a first region positioned in said internal cavity in a first direction, and a second region positioned in said internal cavity in a second direction substantially orthogonal to said first direction.

4. A modular assembly, comprising first and second incubation chambers configured to communicate with each other fluidly, said first incubation chamber comprising a first incubation chamber housing, comprising:
a first inlet port for receiving a fluid,
a first outlet port for dispensing the fluid, and
a first internal cavity;
a first helical coil positioned in said first internal cavity and in fluid communication with the first inlet port and the first outlet port;
a second incubation chamber comprising a second incubation chamber housing, comprising:
a second inlet port for receiving a fluid,
a second outlet port for dispensing the fluid,
a second internal cavity; and
a second helical coil positioned in said second internal cavity and in fluid communication with the second inlet port and the second outlet port;
wherein said first incubation chamber has a top exterior surface having a first contour, and said second incubation chamber has a bottom exterior surface having a second contour, wherein said top exterior surface can releasably interconnect with said bottom exterior surface.

5. The modular assembly of claim 4, wherein said first helical coil is configured to be in fluid communication with said second helical coil when said first and second incubation chambers are interconnected.

6. A method for inactivating one or more viruses in a sample containing a target molecule, wherein the method comprises causing the sample to flow in a helical coil positioned in an internal cavity of an incubation chamber while continuously exposing the sample to inactivation conditions during a process for purifying said target molecule, said incubation chamber having a top exterior surface having a first contour, and a bottom exterior surface having a second contour, wherein said top exterior surface can releasably mate with an incubation chamber having a bottom exterior surface having said second contour, and said bottom exterior surface can releasably mate with an incubation chamber having a top surface having said first contour.

7. The method of claim 4, further comprising subjecting said fluid sample to a Protein A affinity chromatography process, thereby to obtain an eluate; introducing a virus inactivation agent to said eluate, and continuously transferring said eluate to said helical coil and causing said eluate to flow in said helical coil for a time sufficient to inactive said virus.

8. The method of claim 4, further comprising subjecting said fluid sample to an ion exchange chromatography process, thereby to obtain an eluate; introducing a virus inactivation agent to said eluate, and continuously transferring said eluate to said helical coil and causing said eluate to flow in said helical coil for a time sufficient to inactive said virus.
